(19) United States
(12) Patent Application Publication
(10) Pub. No.: US 2008/0154258 A1
(43) Pub. Date: Jun. 26, 2008

(54) Radio Frequency Ablation System with Joule-Thomson Cooler

(75) Inventors: Zhaohua Chang, Shanghai (CN); Peng-Fei Yang, Shanghai (CN)

Correspondence Address: CROCKETT & CROCKETT, 24012 CALLE DE LA PLATA, SUITE 400, LAGUNA HILLS, CA, 92653, US

(73) Assignee: AccuTarget MediPharma (Shanghai) Corp. Ltd.

(21) Appl. No.: 11/935,331

(22) Filed: Nov. 5, 2007

(30) Foreign Application Priority Data
Dec. 26, 2006 (CN) CN200610147978.6

(51) Int. Cl. A61B 18/14

(52) U.S. Cl. 606/41

(57) ABSTRACT

An RF tissue ablation system with a Joule-Thomson cooler for limiting the temperature of the RF electrodes. An RF generator produces electromagnetic energy to ablate the tissue, and may also be used to re-warm the probe when the probe is used as a cryoprobe.

This application claims priority to Chinese Patent Application 200610147978.6 filed Dec. 26, 2006.

### FIELD OF THE INVENTIONS

The inventions described below relate the field of RF ablation.

### BACKGROUND OF THE INVENTIONS

Radio frequency (RF) ablation and cryoablation are widely used for treating many kinds of diseases, including liver tumor, mastadenoma, prostate tumor and cerebroma, etc. Generally, the RF electrode is inserted into the pathological tissue, and a large reference or ground electrode for contracting a large surface of the body is placed on the skin. The high-frequency current passed through the probe tip to the ground electrode heats body tissue in the vicinity of the probe tip, resulting in ablation of the tissue. Cryoablation of diseased tissues is also widely used, accomplished through the application of a cryoprobe to a designated area, which is operated to freeze and thereby ablate a target tissue area.

For RF ablation, the effect of direct thermal ablation is correlated with the temperature achieved within the target tissue, and the temperature is determined by the total thermal energy applied, rate of removal of heat, and the specific thermal sensitivity of the tissue. Generally, heating tissue to a temperature of 42° C. to 45° C. can cause the irreversible cellular damage needed for thermal ablation. The inactivation of vital enzymes within this range of temperature is the most dominant factor in resulting tissue damage. When tissue temperature rises to 60° C., the time of producing irreversible cellular damage is greatly shortened. When the temperature is above 60° C., protein denaturation occurs. An area of coagulation and necrosis block appear. When the temperature continues to rise to about 100° C., water within the tissue is boiled. Even higher temperatures result in carbonization, charring and smoke generation. Once carbonization occurs, the temperature of the target tissue will rise rapidly. Meanwhile, carbonization hinders the tissue further transfer of RF energy into the target tissue, thus limiting the depth of lesions that may be created within the target tissue, and the charring increases the interstitial pressure of tissue, and these effects may cause the cancer cells within the target tissue to spread and penetrate into the tissue and blood vessels.

During the process of RF ablation, the current density is the highest around the electrode, so the temperature in the target tissue is highest immediately proximate the RF electrode. As the distance from the electrode tip increases, the temperature gradually decreases. If the RF ablation energy is improved to increase the temperature, tissue close to the electrode is easy to be charred, making it difficult to create deep lesions.

At present, RF ablation lesion depth is expanded by the following methods: One is to utilize multiple electrodes to increase the diameter of ablation, such as multiple antenna ablation apparatus from Rita Medical Systems, Inc. However, such systems require multiple tissue punctures, and therefore result in additional tissue trauma, and increased danger of damaging adjacent important tissue. Furthermore, the use and operation of the electrodes are complicated, so it is difficult to insert the electrode correctly into the target tissue. In addition, the ablation area of multiple antenna ablation electrode is irregular, so hemorrhage and infection are inevitable.

Another technique for enhancing lesion depth in RF ablation systems is the addition of a cooling element. Ablation electrodes with cooling elements can reduce the probability of carbonization, make more electromagnetic energy applied to the pathological tissue, lengthen ablation time, and finally increase the lesion depth of ablation. For example, the cool-tip electrode of Sherwood Services AG injects fluid coolant, such as water or saline, to reduce tip temperature through heat convection. This system can reduce the excessive temperature of the ablation process adjacent to the tip and increase the heat energy effectively. The cooling element adopted at present is mainly liquid fluid, for instance water, saline, etc. These cooling solutions are pumped into the RF probe to cool the RF electrode. However, owing to the limited size of fluid inlet tube and outlet tube, the flow velocity of cooling solution is relatively slow and the flow is small, so the efficiency of heat exchange is limited. Furthermore, when the temperature of electrode probe is high, the liquid fluid is easy to vaporize and result in vapor lock of the cooling flow.

The nature of the environment created during a cryosurgical procedure results in tissue ablation through several differing mechanisms. Intracellular ice formation and necrosis were originally thought to be the primary causes of cell death. Certain intended destructive effects of this procedure are clear, with freezing resulting in ice formation, and eventual rupture of the targeted cells. The center (closest to the cryoprobe) of the cryogenic lesion is completely necrotic, as temperatures elevate further from the probe tip, solution effects are the primary mechanism cell death. After completion of freezing, warming and/or thawing is initiated. Warming is used to quickly unstick the probe and to thaw the bulk of the frozen tissue. Thawing is a damaging process. The warming for probe extraction is minimally consequential to the tumor mass due to the small zone of tissue affected. The warming to melt the bulk of the frozen tissue can damage the tissue by the mechanisms of solution effects and recrystallization. RF energy provided through an RF electrode in a cryoprobe can be used to thaw the bulk of the frozen tissue and damage the tissue by post-cryoablation warming and by thermal ablation.

### SUMMARY

The RF ablation probe described is combined with a Joule-Thomson cooling system which is operable to cool the RF electrode of the probe in order to prevent overheating of body tissue proximate the probe and enable the creation of larger and deeper lesions. The system can also be operated as Joule-Thomson cryoprobe, wherein the RF electrode can be used to thaw body tissue after cryoablation. This system can control the temperature at the tip of the probe. When operated to accomplish RF ablation, the temperature of tip can be controlled through the modes of RF ablation and cooling, and in this way it can not only create a deep lesion and avoid denaturing tissue adjacent to the RF tip. The gas used for Joule Thomson can be supplied at different pressures to generate different cooling effects, and cooperate with radio frequency energy of different power to change the thermal distribution of the tissue around the probe, in order to control the ablation range. The system includes the probe, handle, transporting tube, control unit and gas container. The control unit can display, control, monitor the parameters of ablation.

### BRIEF DESCRIPTION OF THE DRAWINGS

FIG. 1 shows a first embodiment of a hybrid ablation system with RF ablation and cryogenic cooling modalities.

FIG. 2 shows a cross section of one form of the probe tip.

FIG. 3 is a graph illustrating temperature history with and without the cooling method in the process of radio frequency ablation.

FIG. 4 is a graph illustrating the change of tissue temperature when cooling method is adopted after a certain stage of radio frequency ablation.

FIG. 5 is a graph illustrating temperature distribution associated with the probe in the process of radio frequency ablation.

FIG. 6 is a block diagram illustrating operating methods of the control unit.

### DETAILED DESCRIPTION OF THE INVENTIONS

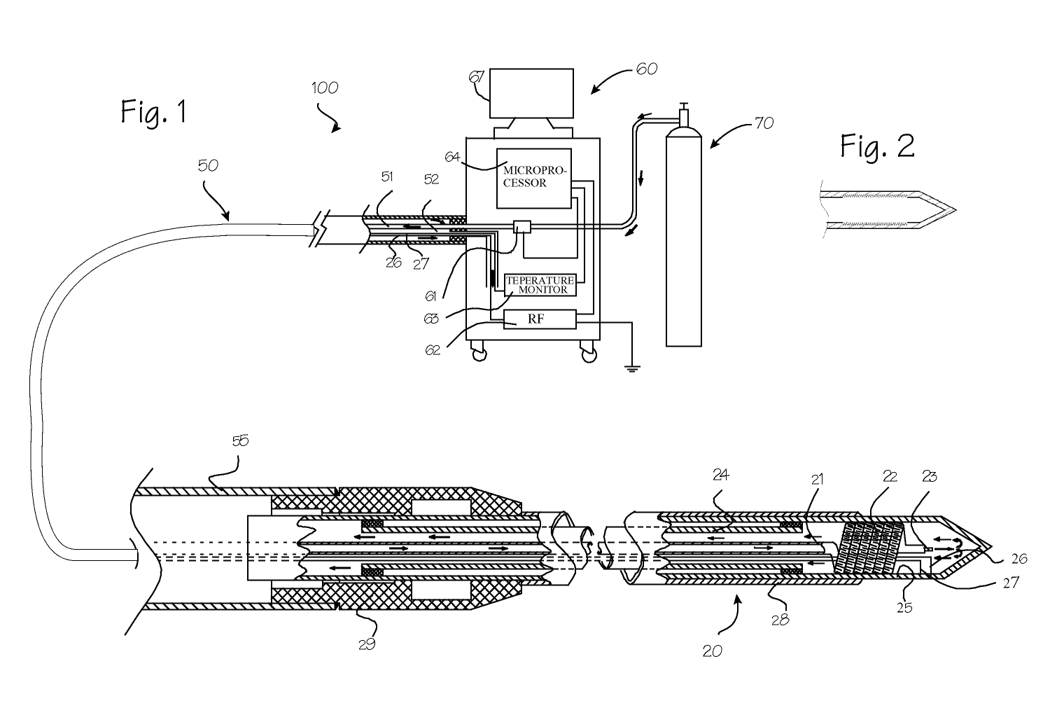

FIG. 1 illustrates a hybrid ablation system 100 with RF ablation and Joule-Thomson cooling modalities and the illustrative elements thereof. The whole system 100 is mainly composed of probe 20 and handle 29, an RF generator 62, high pressure gas reservoir 70, combined RF supply cable and high pressure gas supply line 50, and the control unit 60. Handle 29 and the control unit 60 are connected together through combined RF supply cable and high pressure gas supply line 50. The combined RF supply cable and high pressure gas supply line 50 comprises gas inlet tube 51, gas outlet cavity 52, and RF supply cable 26. There is a microprocessor 64 in the control unit 60, which controls electromagnetic control equipment 61, temperature monitoring equipment 63, RF generator 62 and display equipment 67. The gas inlet tube 51 is linked with gas container 70 through electromechanical control valves 61. Thermo-sensor 27 is linked through cable 53 with temperature monitoring equipment 63, wherein RF line 26 is linked with RF generator 62.

As shown in FIG. 1, in the probe 20, gas flows in high-pressure tube 21 and spiral finned tube 22 to Joule-Thomson nozzle 23. When supplied with high pressure gas, such as Argon, Joule-Thomson effect leads to cooling of the gas upon exit from the nozzle. The lumen on the tip of the probe is filled with the cooling gas, and the gas cools the probe wall, and upon exhaust also cools the spiral finned tube 22 and probe wall, then discharges through the lumen between heat insulation tube 24 and high-pressure tube 21, and vents to atmosphere at the bottom of the control unit 60 through gas supply line 50.

The tip 25 of probe 24 is adapted for easy insertion into pathological tissue. It comprises an outer sheath with a closed distal end. The length and diameter of the sheath is selected depending on the size of pathological tissue to be ablated, and is inserted into the tissue to a depth such that the RF electrode is located within the pathological tissue. The outer sheath may comprise stainless steel, nickel titanium alloy or titanium, etc. As shown in FIG. 2, the inner surface of the tip 25 may be fitted with internal screw fins for a length of about 2 cm to 3 cm. This facilitates heat exchange between the cooling gas and the probe tip and cooling of the external wall of the probe.

In the probe 20, RF line 26 is connected with the tip by junction (a weld, braze, or other secure electrical connection). RF power supplied by the RF generator 62 is transmitted through the pathological tissue, between the tip and a reference ground or indifferent electrode, to heat the pathological tissue to temperature sufficient to cause ablation. The heating of the tissue can be controlled through controlling the power of RF generator 62.

The elongated tissue-penetrating probe includes an insulating coating 28 in order to prevent the flow of electric current from the shaft of the probe into the health tissue surrounding proximal portions of the probe. Therefore, except the tip of probe, surrounding tissue contacting with the shaft of probe is not heated up. The length of insulating coating can be changed to alter the effective length of the probe from which ablative energy will pass into body tissue.

The ablation temperature of the tip of probe 25 can be adjusted through the cooling effect generated by gas passing through Joule-Thomson nozzle 23, thus the temperature of the tissue in contact with probe can be controlled. In the embodiment shown in FIG. 1, gas flows into spiral finned tube 22 and then exits the Joule-Thomson nozzle 23. The pressure sharply drops after the gas flows through Joule-Thomson nozzle 23, and this results in cooling of the gas to cryogenic temperatures. Lumen on the tip of the probe is filled with the cooling gas, and the cooled gas exhausts over spiral finned tube 22 and pre-cools incoming gas through heat exchange with spiral finned tube 22, to enhance the cooling effect. This classic fin-tube helical coil heat exchanger is preferred, but other heat exchange arrangements may be used, including a straight fin-tube counterflow heat exchanger, or a spiral-finned counterflow heat exchanger.

The gas used in system 100 is the gas having a positive Joule-Thomson effect, such as nitrogen, argon and most other gases. The gas is stored in gas reservoir 70. Gas container 70 has a certain initial pressure, such as 1800 psi. The pressure of gas can be controlled by electromagnetic control equipment 61. The different cooling capacities can be produced under different pressures of supplied gas. The control system is operable to alter the supplied gas pressure, through pressure control valves in the electromagnetic control equipment, to effect different levels of cooling. Therefore, temperature probe tip and of the surrounding tissue can be controlled or changed through changing and balancing the gas pressure supplied to the probe tip and RF power supplied to the RF electrode in the tip. The cooling can reduce the temperature of tissue in contact with the tip of the probe 25 to avoid necrosis and/or charring of the tissue, so that RF energy supplied through the tip can be applied without regard to the high electrical resistance of necrosed and charred tissue.

Thermo-sensor 27 in the probe 20 may be thermocouple, thermal resistance or sensors of other forms. The signal gathered by the sensor indicates the temperature of surrounding tissue or the degree of ablation. The temperature monitoring equipment 63 and microprocessor 64 process the temperature signal provided by the thermo-sensor and control the RF generator 62 and electromagnetic control equipment 61 to achieve a desired ablation profile.

In the lumen of the probe 20, heat insulation tube 24 is disposed coaxially between the outer sheath and the gas inlet tube 21. It extends through the probe 20 and both ends of it are fixed to the inner wall of probe by soldering or other means, to create an air insulated or vacuum insulated chamber proximal to the distal tip of the probe. The heat insulation tube can comprise stainless steel or other materials. When the cooling gas in the front of the probe is flowing out, heat insulation tube 24 and air chamber can prevent the cold gas from contacting the probe wall to protect healthy body tissue contacting with the shaft of the probe 20 from the influence of cold gas.

Handle 29 is a hollow tube which provides an ergonomic handle structure and serves as a support structure for joining the several components of the probe. The end of the probe 20 fits tightly into the distal end of the handle. The proximal end of the handle fits tightly into outer tube 55 of high pressure gas supply tube. The handle can be made of any material, an is preferably made of an thermally and electrically insulative material.

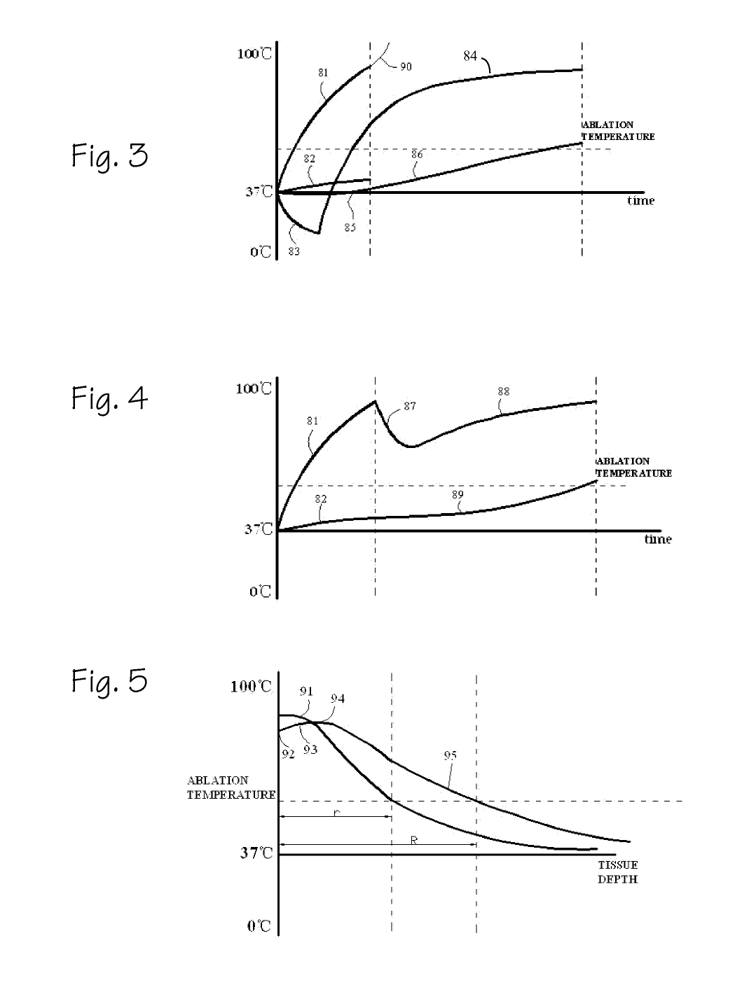

To consider temperature distribution from the tip, reference will be made to the graph of FIG. 3. This graph illustrates temperature history during RF ablation, both with and without application of cooling gas. It shows the curves of tissue temperature changing with time. The horizontal axis corresponds to ablation time, while the vertical axis corresponds to tissue temperature. Body temperature of 37° C. is indicated by the horizontal solid line. Also, a temperature level of 100° C. is marked. It has mentioned in the above, 100° C. is the boiling point of water and is very important in the course of ablation of tissue, and body temperature is easily charred when the temperature exceeds 100° C. A temperature level of 0° C. (the freezing point of water) is also marked in the graph. It is generally accepted that cell damaging temperature in tissue begins in the range of 42° C. to 45° C. Therefore, temperatures in this range can be regarded as the ablation temperatures, as indicated in the figure by the dashed line. There is no scale unit in the figure and the curve shows the general trend of the change of tissue temperature. This has been found effective to provide mapping an ablation lesion.

Curves 81 and 82 in FIG. 3 show that when traditional RF ablation probe is used, the temperature history of tissue close to the ablation probe (Curve 81) and tissue farther from the ablation probe, for example about 2 cm from the probe (Curve 82). When the RF ablation begins, it can be seen from curve 81 that the temperature of tissue close to the ablation probe rises rapidly and exceeds the ablation temperature in a short time, and can quickly reach 100° C. Curve 82 shows that within this period of time, the temperature of tissue farther from the ablation probe rises slowly and does not reach the ablation temperature, so the effect of ablation in this place is limited. If RF heating proceeds, curve 81 will extend to curve 90. At this moment, temperature rises rapidly and exceeds 100° C., which results in the charring of tissue and increasing of resistance, therefore the ablation process is stopped. If RF power is reduced to continue the RF course, the temperature of tissue farther from the ablation probe cannot be raised yet and cannot reach the effect of ablation. The depth of lesions achievable in this situation is relatively small.

Temperature curves represented by curves 83 and 85 illustrate the characteristic temperatures in tissue near and distant from the probe of FIG. 1 when operated to provide RF ablation with JT cooling. Before the RF heating, electromagnetic control equipment 61 make the gas flow into the probe 20, and Joule-Thomson effect occurs at the tip of the probe to cool the tissue close to the electrode probe. Curve 83 shows that the temperature drops to a certain temperature, which depends on the time and the gas pressure. Tissue farther removed from the ablation probe is not affected and remains at 37° C., as curve 85 shows. Subsequently, RF ablation starts and the temperature rises gradually. The change can be seen in curve 84. Because the cooling gas sufficiently exchanges heat with the wall of the probe, the temperature of tissue contacting with the probe wall changes slowly after rising to ablative temperatures. Therefore, it is hard to exceed 100° C. and cause charring of the tissue that would impede transfer of RF energy. However, because the mechanism of RF heating and cooling is different, the cooling effect of gas does not influence the tissue farther from the ablation probe. The temperature of tissue remote from the probe tip continues to rise due to the passage of RF energy through the tissue. It can be seen in curve 86 that the temperature of tissue spaced from the probe tip (again, at about 2 cm from the tip) reaches and exceeds the ablation temperature in time. This illustrates that the range of ablation increases greatly when adopting this improved RF ablation system.

FIG. 4 shows the change of tissue temperature when cooling method is initiated after a period of radio frequency ablation. The course of curve 81 and 82 has already been described in FIG. 3. When the radio frequency heating temperature is close to 100° C., such as 80° C., gas flow is initiated to cool the probe tip and immediately adjacent tissue to prevent the charring of tissue. It can be seen in curve 87 that temperature at or near the probe may drop upon initiation of cooling flow. The relative amounts of RF power and cooling gas supplied to the probe can be adjusted to provide heating power greater than the cooling power, so that the temperature of the tissue close to the probe remains above the ablation temperature and begins to rise after dropping, is illustrated by curve 88. The temperature of the tissue farther from the probe slowly rises and eventually exceeds the ablation temperature. FIGS. 3 and 4 show the different methods, in which cooling carried on at different times relative to the start of the RF ablation process effects the temperature profile of the tissue surrounding the probe. The cooling gas flow can be started and stopped for many times according to the actual conditions of the body tissue, as indicated by the temperature sensor in the probe, and the gas pressure can be changed to adjust the course of ablation.

FIG. 5 shows the curve of the tissue temperature changing with the distance of tissue from the probe. The nominal radial distance TISSUE DEPTH from the central axis of a probe tip is plotted against temperature T. The ablation temperature is marked in the figure, namely ABLATION TEMPERATURE indicated by the dashed line. The corresponding distance of ABLATION TEMPERATURE is the ablation radius or tissue depth. The curve 91 represents the operation of a traditional ablation probe. It can be seen that the temperature rapidly falls off and approaches body temperature a short distance from the probe. When the method combining cooling with radio frequency has been carried on for a period of time, the temperature of the tissue close to the probe surface is illustrated by point 92 and this temperature is higher than the ablation temperature. Because the cooling power is relatively small, it is difficult to conduct the energy away from (i.e., cool) the tissue farther from the probe, but the current density here is still large. Accordingly, as shown by curve 93, the temperature rises slowly. With the increase of distance, the volume of the tissue ablated becomes bigger, and conduction and heat convection become serious, so the temperature begins to drop gradually after reaching the peak 94, as shown by curve 95. Because the cooling provides for greatly lengthened ablation time, the temperature of tissue farther from the electrode probe reaches the ablation temperature as well. In this case, ablation radius increases to R, obviously far larger than the ablation radius r achieved without cooling. This improved system can not only be widely used in treating various kinds of tumors owing to its larger ablation diameter, which take the place of using several traditional RF ablation probes, but also be used effective for rewarming the freezing tissue in the process of cryoablation.

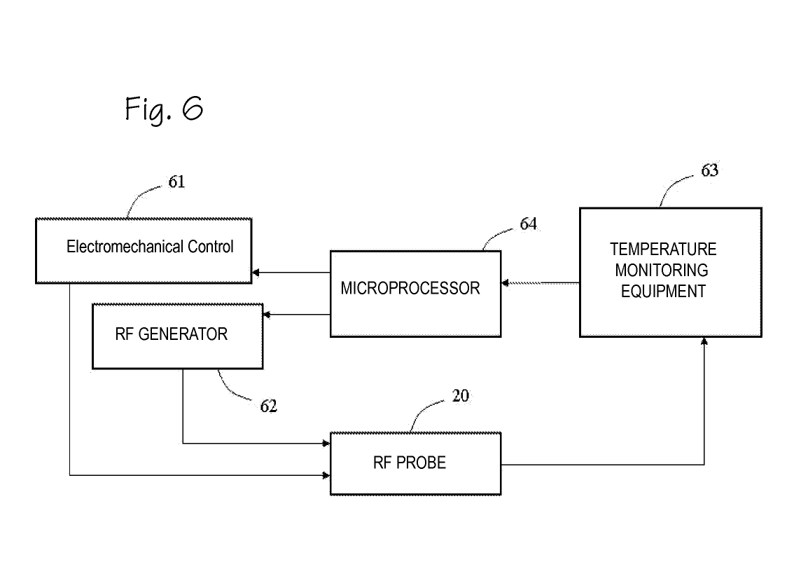

FIG. 6 is a block diagram of the control unit of this hybrid ablation system with RF ablation and cryogenic modalities. It can be seen from the FIG. 8 that the RF generator 62 applies the electromagnetic energy to the tissue to be ablated through the probe 20. Temperature monitoring equipment 63 monitors the temperatures of the probe 20, and provides signals indicative of the temperature to microprocessor 64. If the temperature of the probe is high, microprocessor 64 will send a signal to electromechanical control system. electromechanical control system 61 will function, as directed by the microprocessor, to provide cooling gas at suitable pressure to the probe, and then the temperature of the probe comes down. If the temperature of the probe is low, the temperature of the probe tip will be controlled by increasing the generating power of RF generator 62 to realize ablation. In this way, the temperature can be regulated and controlled to carry on different treatment schemes.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A RF ablation system for tissue ablation, comprising:
an RF ablation probe comprising rigid tube with a closed distal end adapted for insertion into the body of a patient, said probe having a distal tip with an electrically and thermally conductive outer surface, and an RF conductor in electrical communication with the closed distal end of the rigid tube, for deliver of RF energy to the body of the patient through the distal end of the rigid tube;
an electrically and thermally insulating sleeve or coating disposed over the rigid tube, proximal to the distal end of said rigid tube
a Joule-Thomson cooler comprising a counter-flow heat exchanger disposed within the rigid tube, with an outlet in communication with the space within the closed distal end of the rigid tube;
a thermo-sensor disposed within the distal end of the rigid tube,
a reservoir of high pressure cooling gas aligned to supply cooling gas to the Joule-Thomson cooler;
a second insulating sleeve disposed with the rigid tube, said insulating sleeve providing an exhaust pathway for cooling gas exiting the Joule Thomson cooler.
means for controlling cooling gas flow to the Joule-Thomson cooler and delivery of RF energy to the ablation probe, in order to limit temperature at the surface of the probe while delivering RF energy into the body of the patient.
